(19) United States
(12) Patent Application Publication
(10) Pub. No.: US 2010/0081949 A1
(43) Pub. Date: Apr. 1, 2010

(54) ECG LEAD-OFF DETECTION USING PHASE SHIFT OF RECOVERED TRANSTHORACIC IMPEDANCE RESPIRATION SIGNAL

(76) Inventor: William J. Derby, JR., Bethlehem, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/239,549

(22) Filed: Sep. 26, 2008

(51) Int. Cl. A61B 5/0402

(52) U.S. Cl. 600/509

(57) ABSTRACT

A lead-off detector is disclosed for detection of a change of connection state of one or more of the electrical leads which are used to connect a physiological monitoring device, such as an Electrocardiogram (“ECG”) monitoring device, with the subject, e.g. patient, being monitored. The disclosed lead-off detector detects such changes rapidly and is not sensitive to electrical noise or thermal fluctuations. For example, the disclosed embodiments may detect disconnection and/or connection of one or more electrical leads and may further detect connection and/or disconnection of one or more electrical leads from the monitoring device, the subject, intervening coupling devices, or a combination thereof. In one embodiment, the disclosed lead-off detector is used in conjunction with a three lead ECG monitor with Transthoracic Impedance Respiration (“TIR”) functionality but may also be utilized with other types of physiological monitoring devices. The disclosed lead-off detector measures a phase difference between a signal transmitted to the subject and a signal received therefrom. Wherein the electrical leads are properly connected, the phase difference will be below a defined threshold. However, when one or more of the electrical leads becomes disconnected in some manner, the phase difference between the transmitted signal and the received signal will exceed the threshold. In an alternate embodiment, the clock frequency of the transmitted signal is further compared with the clock frequency of the received signal to detect transitions between the lead connection states and indicate a lead-off event so as avoid noise generated therefrom, referred to as “lead noise”, from being further processed.

### BACKGROUND

In order to properly process electrocardiogram (“ECG” or “EKG”) and/or respiration physiological signals from a body, e.g. patient, monitored by a physiological monitoring device, such as an ECG monitoring device (sometimes referred to as an “elecrocardiogram” or “electrocardiograph” device or generator, or combination or derivation thereof), it is vital to know when the electrical leads, which couple the monitoring device with the body of the subject/patient, are attached or not attached to the body. Failure to detect this state of attachment, referred to as “lead-off” detection, may cause spurious triggering of other devices connected to the monitoring device and which perform various functions based on the monitored ECG of the subject, as well as delayed recovery of proper signals when the leads are attached to the subject.

Generally, an ECG monitoring device uses several electrical leads to couple the monitoring device with the subject and measures the ECG signal using the resistance between these leads, one of which is typically placed on the chest by the left arm (LA) of the subject and one typically placed on the chest by the right arm (RA). A third lead (RL) is usually placed on the leg or lower abdomen and provides a “ground” reference for the two signals. Thereby, electrical activity of the subject's heart may be monitored. In addition, other physiological signals, such as respiration, may be monitored simultaneously using the same leads using, for example, Transthoracic Impedance Respiration or TIR, also referred to as Impedance Pneumography. The TIR method injects a high frequency signal (Typically a 100 Khz sinusoidal wave—well above other physiological signal frequencies of interest, such as the ECG signals, so as not to interfere therewith) on the LA lead and then measures the signal received on the RA lead. Physical changes in the body as the subject inhales and exhales are reflected in changes in the attenuation of received signal. While ECG monitoring devices may include respiration monitoring capability, it will be appreciated that this function may be performed by a separate physiological monitoring device or respiration monitoring functionality may be include in another type of monitoring device.

The respiration signal in this scenario is typically produced by taking the average of the positive and negative halves of the recovered waveform and then low-pass filtering the averages into a slowly changing output. For example, a 4'th order Butterworth low-pass filter with a corner frequency of 100 Hz and a gain of 22 may be used. This signal is digitized and passed into a digital signal processor (“DSP”) where gain stages and additional filtering is performed to extract the frequencies in the respiration range—typically 0.16 Hz to 3 Hz along with a direct-current (“DC”) component (below 0.16 Hz) for lead off detection.

The positive and negative halves of the received wave are detected using a clock recovery circuit because the TIR signal is coupled in and out of the ECG circuit using transformers (for isolation) and the subject's body also has (small) reactive characteristics. These reactive components shift the receive waveform compared to the transmitted wave. Therefore, to properly find the zero crossings on the recovered wave—necessary for accurate averages—a clock recovery circuit is employed to find and produce a square wave output corresponding to the positive and negative. This signal is used to drive the circuit that finds the average of the waveform.

Present methods for lead-off detection are based on the idea that when the leads are off, e.g. because one or more of the leads became disconnected from the subject or the monitoring device, etc., the resistance between LA and RA signals becomes extremely high, reducing the amplitude of the recovered signal and hence the respiration signal. The DC component (<0.01 Hz) of the respiration signal is compared to a threshold and if the signal is below the threshold, then a lead-off situation is said to occur.

Unfortunately, this resistance based method of lead-off detection suffers from numerous problems. In particular, the method suffers from a slow response time. The high amount of noise and low frequency of the respiration signals of interest (in this case the DC component) requires filters with a significant amount of time delay. This means that when the leads are removed, there is significant time required for that change to propagate to the point where the lead off threshold can be measured. This same situation applies to the lead-on transition. In addition, the resistance based method of lead-off detection exhibits thermal sensitivity. The high gain and low level of the recovered signals makes this circuit particularly sensitive to temperature changes. This sensitivity is generally due to changes in resistance vs. temperature of the circuit's resistors, including the resistor that provides “constant current” into the transmit side and the various op-amp resistors in the analog op-amp filters prior to being digitized. Note that these temperature based movement do not affect the monitoring of respiration itself because the 0.16 Hz high-pass filtering removes the DC content and the temperature changes are well below the 0.16 cutoff. The lead-off processing, however, is sensitive to these movements. Typically, the DC component moves so much that no single lead threshold can be used reliably unless a temperature sensor is added to normalize the results.

Further, prior lead-off detection methods based on resistance are subject to interference from signal noise depending upon the type of lead-off situation which has occurred. The leads that couple the monitoring device with the subject are constructed of several parts including connectors which connect the leads to the monitoring device and mechanisms for connecting the other end of the leads to the subject's body, including snaps-connectors and disposable adhesive electrodes/patches, etc. Specifically there may be at least 3 “lead off” scenarios with some sub-cases within the last. These cases are controlled by the physical construction of the lead, such as an Association for the Advancement of Medical Instrumentation (“AAMI”) ECG cable—which has a standard connector (DIN type) the end that plugs in the machine and on the other end a set of color coded sockets. This cable has a specified resistance, e.g. 1K Ohm. To this connector are attached 3 color coded snap leads (LA, RA, RL) which are single wires with a plug at one end to mate with the cable and a female snap end designed to connect to the electrodes (male snap) that are placed on the body typically with an adhesive patch. Depending upon which part of the lead becomes disconnected, the resistance measured may vary necessitating a different threshold to determine that a lead-off has occurred.

### BRIEF DESCRIPTION OF THE DRAWINGS

FIG. 1 depicts a block diagram of a ECG monitoring device having a lead-off detector according to one embodiment.

FIG. 2 depicts a more detailed block diagram of the monitoring device of FIG. 1.

FIG. 3 depicts a more detailed block diagram of the lead off detector for use with the monitoring device of FIG. 1.

FIG. 4 depicts a flow chart demonstrating operation of the monitoring device of FIG. 1 according to one embodiment.

### DETAILED DESCRIPTION OF THE DRAWINGS AND PRESENTLY PREFERRED EMBODIMENTS

The disclosed embodiments relate to a lead-off detector for detection of a change of connection state of one or more of the electrical leads which are used to connect a physiological monitoring device, such as an Electrocardiogram (“ECG”) monitoring device, with the subject, e.g. patient, being monitored. The disclosed lead-off detector detects such changes rapidly and is not sensitive to electrical noise or thermal fluctuations. For example, the disclosed embodiments may detect disconnection and/or connection of one or more electrical leads and may further detect connection and/or disconnection of one or more electrical leads from the monitoring device, the subject, intervening coupling devices, or a combination thereof. In one embodiment, the disclosed lead-off detector is used in conjunction with a three lead ECG monitor with Transthoracic Impedance Respiration (“TIR”) functionality. It will be appreciated that the disclosed functionality may be utilized with other types of physiological monitoring devices. As will be described in more detail below, the disclosed lead-off detector measures a phase difference between a signal transmitted to the subject and a signal received therefrom. Wherein the electrical leads are properly connected, the phase difference will be below a defined threshold. However, when one or more of the electrical leads becomes disconnected, the phase difference between the transmitted signal and the received signal will exceed the threshold. In an alternate embodiment, the clock frequency of the transmitted signal is further compared with the clock frequency of the received signal to detect transitions between the lead connection states and indicate a lead-off event to avoid noise generated therefrom, referred to as “lead noise”, from being further processed.

As was described above, prior lead-off detection methods based on resistance are subject to interference from signal noise depending upon the type of lead-off situation which has occurred. The leads that couple the monitoring device with the subject are constructed of several parts including connectors which connect the leads to the monitoring device and mechanisms for connecting the other end of the leads to the subject's body, including snaps-connectors and disposable adhesive electrodes/patches, etc. Specifically there may be at least 3 “lead off” scenarios with additional variation thereof. The various scenarios derive from the physical construction of the electrical lead, such as an Association for the Advancement of Medical Instrumentation (“AAMI”) ECG cable—which has a standard connector (DIN type) on one end that plugs into an interface of the monitoring device and a set of color coded sockets on the opposite end. This cable has a specified resistance, e.g. 1K Ohm. To this connector are attached 3 color coded snap leads (LA, RA, RL) which are single wires with a plug at one end to mate with the cable and a female snap end designed to connect to the electrodes (male snap) that are placed on the body typically with an adhesive patch. Depending upon which part of the lead becomes disconnected, the resistance measured may vary necessitating a different threshold to determine that a lead-off has occurred. For example, the following lead-off scenarios may occur:
No Cable attached;
1K Cable but no snap leads; or
1K Cable with snap leads, not attached to the body.
(subgroups of one or more leads not attached to a body, e.g. LA on, RA & RL off.

As used herein, the term “lead” or “electrical lead” refers interchangeably to the individual wires used to convey the LA, RA and RL signals and the collection thereof into a bundled cable, i.e. a cable which features a single connector on one end for connection to the monitoring device and individual connections on the opposite end for connection to the LA, RA and RL locations on the subject, such as via adhesive electrode patches. The disclosed embodiments relate to detection of an interruption of the electrical connection created between the monitoring device and subject by the leads, physical implementation of which are implementation dependent.

The disclosed embodiments detect a lead-off situation based on a pronounced phase shift between the recovered and transmitted signal when a lead becomes disconnected, this shift typically being far in excess of the shift typical for connection on a normal body—(where resistive coupling provides a much stronger return signal and reduced reactance). As such, the disclosed embodiments detect disconnection under any of the above scenarios.

As will be discussed in more detail below, the disclosed embodiments augment a standard monitoring device, e.g. a standard ECG monitoring device or other diagnostic medical device having physiological monitoring capabilities, such as a diagnostic medical ultrasound system which provides for ECG and respiration monitoring capabilities in addition to imaging capabilities, by adding a digital phase detector to detect a phase shift between the transmit and receive signals. In one embodiment, the digital phase detector is implemented using a high speed counter, for example counting at 60 MHz, which is started upon receipt of the leading edge of the transmitted clock and stopped by the leading edge of the clock signal recovered from the receive signal. Alternatively, a recovered transmitted clock may be used to start the counter, i.e. the clock signal derived from the actual signal transmitted to the subject, or an approximation thereof, rather than the transmitted clock directly. Using the recovered transmitted clock may improve accuracy by eliminating variations in the signal introduced by various circuits which generate the transmitted signal based on the transmitted clock, such as noise or temperature variations, e.g. phase shifts, etc. In yet another alternative embodiment, the recovered transmit clock is inverted and the inverted recovered transmit clock and the recovered receive clock may be logically AND'ed together before being passed to the counter wherein the counter is started by a first leading edge of the combined clock signal and stopped by the next trailing edge. Using a combined clock signal reduces the number of signals which need to be passed to the phase detector (counter and comparison logic)§ making implementation easier where circuit and/or interconnection resources are limited. Essentially, this counter determines a measurement of the relative phase between the transmit and receive clocks. Further, since this measurement is made every cycle of the transmit clock waveform, the phase change detection is nearly instantaneous. In addition, since the only analog component in the lead-off detector of the disclosed embodiments is the clock recovery circuit for the receive signal—the operation of which is not highly sensitive to temperature—the detection operation is generally thermally insensitive and accurate to the limits of the high speed counter, e.g. 60 MHz in one embodiment. The detector may even be insensitive to changes in the transmit drive current, particularly when utilizing the recovered transmit clock, because the phase shift is a characteristic of the inductive coupling—not the power transferred through it. As can be seen, the disclosed lead-off detector takes advantage of the properties which cause the problems in the prior lead-off detectors.

In an alternative embodiment, an additional counter is added to measure the duration of the recovered clock from the receive signal to verify that the recovered clock is stably detecting the same frequency clock signals as transmitted, in one embodiment a 100 Khz signal. During lead connection transitions, e.g. the time that the lead is losing contact (or gaining contact in situations where the lead is being applied) there is a lot of signal bouncing back and forth between states which produces “lead noise,” i.e. unwanted spikes in the ECG and Respiration signals. Further, the recovered clock of the receive signal will also tend to deviate from the transmit clock frequency, e.g. 100 Khz, as it reacts to the phase changes. By verifying that the recovered clock waveform has the same frequency, e.g. 100 Khz, as the transmit signal prior to looking for the phase difference, lead noise events can be prevented from entering further signal processing. This permits substantially instantaneous lead-off detection even if there was no reactive coupling between the leads because, in a lead-off situation, the recovered receive clock would no longer be stable at the transmit frequency. In embodiments utilizing a combined transmit and receive clock signal, the appropriate frequency may be detected by measuring the elapsed time between consecutive leading edges of the signal.

In one embodiment, the disclosed lead-off detector is implemented as part of a physiological monitoring component of a diagnostic medical ultrasound system using an on-board FPGA to implement the counters and the on-board Digital Signal Processor (“DSP”) to implement the detection logic. The counters accumulate multiple sets of measurements, e.g. 16 sets, before being transferred to a register accessible by the DSP to match the signal processing rate thereof. Other accumulations, such as 10 sets, may be used and are implementation dependent, in particular for balancing response speed versus sensitivity to noise. It will be appreciated that disclosed embodiments may be implemented in hardware, software or a combination thereof and that the counters and detection logic may implemented using other types of processors, etc. Furthermore, the functions described herein may be performed by one or more components, or the described components may be further sub-divided, and is implementation dependent.

In one embodiment, the DSP is suitably programmed to evaluate the counter values and determine when a lead-off event occurs. For example, the DSP may be suitably programmed according to the following pseudo code:

| TIR_Phase = *TIR_PHASE_COUNT (FPGA Register) | |
|---|---|
| width | = TIR_CLOCK_COUNT (FPGA Register) |

// variation from 100Khz (9600 cycles at 60Mhz)
Width_Deviation = absolute value of (9600 − width)

// compare the phase info to the threshold

```
  if ((Width_Deviation > 50) or (TIR_Phase > Threshold)) then
    Set_Leads_off
  Else
    Set_Leads_on
  End If
```

The described lead-off detection mechanism provides extremely fast and reliable lead-off detection. By suspending signal processing and trigger detection during lead off/lead noise events the typical trigger and signal recovery time has been greatly reduced.

To clarify the use in the pending claims and to hereby provide notice to the public, the phrases “at least one of <A>, <B>, . . . and <N>” or “at least one of <A>, <B>, . . . <N>, or combinations thereof” are defined by the Applicant in the broadest sense, superceding any other implied definitions herebefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

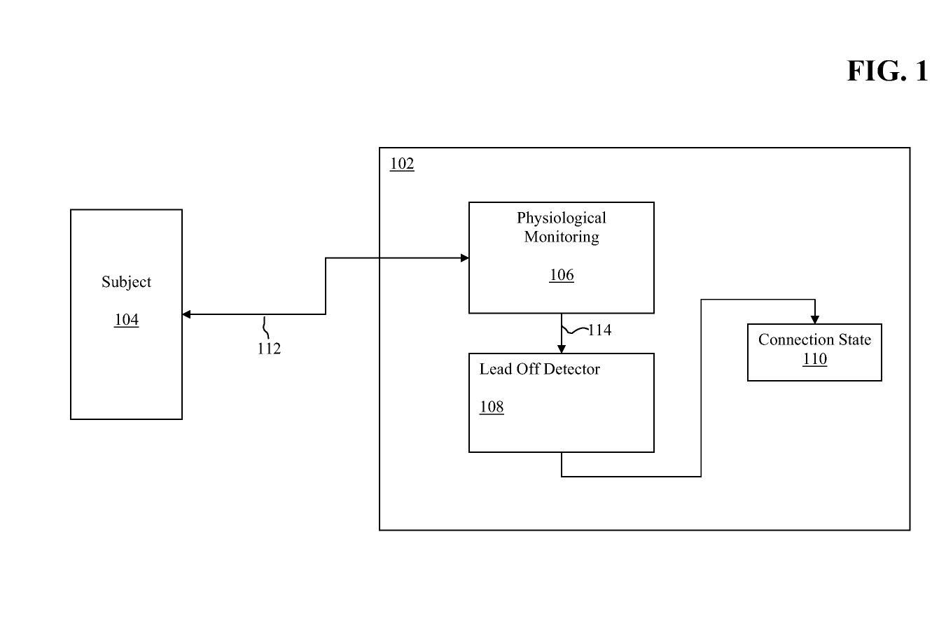

Referring to FIG. 1, there is shown a physiological monitoring system 102, such as an ECG monitoring device, having a lead-off detector 108 according to one embodiment. The monitoring system 102 includes a physiological monitoring device 106 for monitoring one or more physiological parameters of a subject 104 via a set of electrical leads 112. The physiological monitoring device 106 may include any suitable monitoring device such as an ECG monitoring device which further uses the TIR method for monitoring respiration parameters of the subject. The physiological monitoring device 106 is further coupled with the lead off detector 108. Herein, the phrase “coupled with” is defined to mean directly connected to or indirectly connected through, or integrated with, one or more intermediate components. Such intermediate components may include both hardware and software based components.

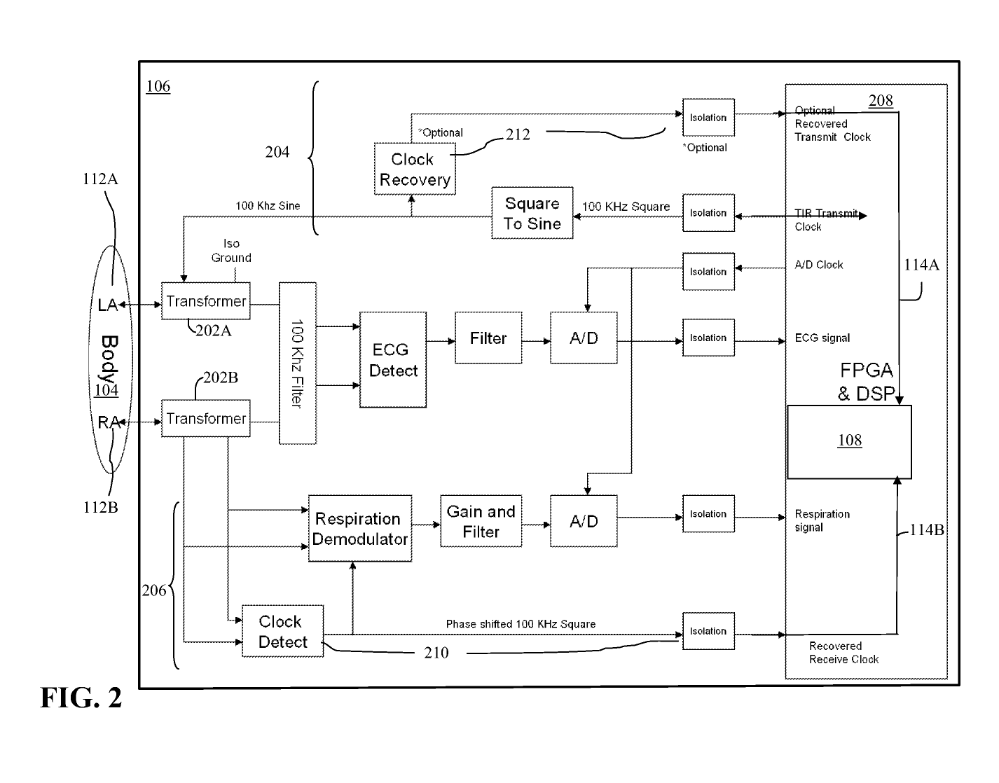

FIG. 2 depicts a more detailed block diagram of the physiological monitoring device 106 of FIG. 1 having the lead off detector 108 integrated therewith. The lead off detector 108 detects interruptions in the electrical connection between the monitoring component 108 and the subject 104 being monitored. The monitoring device 106 includes a first interface 202A, such as an electrical connector to which a lead is connected, operative to couple the physiological monitoring device 106 with a first electrical lead 112A, e.g. the LA lead, to be further coupled with the subject 104, such as via a snap lead and adhesive electrode (both of which are not shown). The physiological monitoring device 106 further includes a second interface 202B operative to couple the physiological monitoring device 106 with a second electrical lead 112B, e.g. the RA lead, to be further coupled with the subject 104, such as via a snap lead and adhesive electrode (both of which are not shown). The first interface 202A, first electrical lead 112A, second interface 202B and second electrical lead 112B forming an electrical connection between the physiological monitoring device 106 and the subject 104.

The monitoring device 106 further includes a transmitter 204 operative to transmit a transmit signal, such as a 100 KHz sinusoidal waveform, via the first interface 202A for transmission to the subject 104 via the first electrical lead 112A, e.g. the LA lead, the transmit signal being characterized by a transmit phase. Under normal operating conditions, the transmit signal is conveyed to the subject 104 by the first electrical lead 112A from the first interface 202A. However, this may not be the case if, for example, the first electrical lead 112A becomes, or is in the process of becoming, disconnected from either the subject 104 or the interface 202A. The monitoring device also includes a receiver 206 operative to receive a receive signal via the second interface 202B, the receive signal being characterized by a receive phase. Under normal operating conditions, the receive signal is received by the second interface 202B from the subject 104 via the second electrical lead 112B, e.g. the RA lead. However, this may not be the case if, for example, the second electrical lead 112B is presently disconnected or in the process of being disconnected from either the subject 104 or the monitoring device 106.

The monitoring device 106 further includes a processor 208, such as a digital signal processor, which processes the received signals to derive ECG, respiration or other physiological signals as is known.

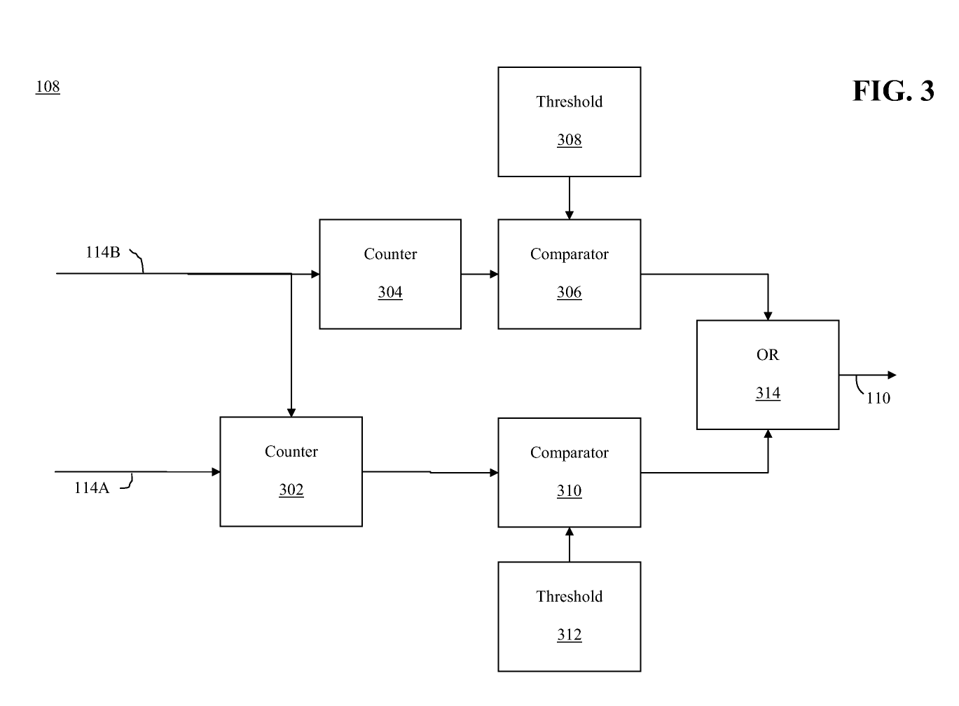

FIG. 3 depicts a more detailed block diagram of the lead off detector 108 for use with the monitoring device of FIG. 1. In one embodiment, the lead off detector 108 is implemented primarily in software which is executed by the processor 208. It will be appreciated that the disclosed functionality may be implemented in hardware or in software using an independent processor which is not shown. The processor 208 is coupled with the transmitter 202A and the receiver 202B. The processor is further coupled with a receive clock detector 210, shown in FIG. 2, which is operative to recover the clock component of the receive signal and provide it to the processor 208. The receive clock detector 210 may be implemented as a comparator, such as a LM311 comparator manufactured by National Semiconductor Corporation, located in Santa Clara, Calif., a phase-locked loop (“PLL”) or other known circuit for accurately deriving a square-wave clock signal from an analog signal at the frequency of interest.

The processor 208 is operative to determine a first difference, i.e. a phase difference, between the transmit phase of the transmit signal and the receive phase of the receive signal. In one embodiment, the processor 208 is further coupled with a transmit clock detector 212, shown in FIG. 2, similar to the receive clock detector 210 which is operative to recover the clock component of the transmit signal and provide it to the processor 208. Alternatively, in implementations where the processor 208 itself is generating the transmit clock signal, this signal can be directly provided to the lead-off detection functionality. In an alternate embodiment, the receive clock signal and transmit clock signal, recovered or as generated, may be logically combined, such as with an AND function and the combined clock signal utilized by the processor to start and stop the counter 302 as further described. In one embodiment, the processor 208 is further operative to determine the first difference by counting, with a counter 302, a number of time intervals elapsed between a leading edge of the transmit signal 114A, i.e. the clock signal/square-wave recovered therefrom, and a leading edge of the receive signal 114B, i.e. the clock signal/square-wave recovered therefrom, the first difference comprising the counted number of time intervals elapsed. As described above, in one embodiment, the counter 302 is a 60 MHz counter which is started by the leading edge of the transmit signal 114A and stopped by the leading edge of the receive signal 114B. Alternatively, in implementations using a combined clock signal, a first leading edge may be used to start the counter 302 and a subsequent trailing edge may be used to stop the counter 302. The processor 208 compares the first difference with a first threshold 312 using a comparator 310, or similar logic, and determines that the electrical connection between the physiological monitoring device 106 and the subject 104 has been interrupted when the first difference deviates from, e.g. exceeds or falls below, the first threshold 312. In one embodiment, the first threshold is 2.1291 microseconds, expressed as the counts of the counter, e.g. at 60 MHz, divided by the average events, e.g. 16 (125.75 average counts at 60 MHz is approximate 2.1291 ms) but is implementation dependent and may be set/calibrated, for example, based on operation with a simulated resistance, such as 5 KOhm to simulate a body plus the leads, between the leads plus an added offset for manufacturing variations in the system components.

In an alternate embodiment, the lead off detector 108 further includes a receive clock frequency detector (not separately shown) which validates that the frequency of the receive clock signal is within a certain margin of the transmit clock frequency. As was discussed above, if the receive clock frequency is out of range, this also indicates a lead-off condition, either an actual disconnect or a disconnect in process. Wherein the transmit signal is further characterized by a transmit clock frequency and the receive signal is further characterized by a receive clock frequency, the processor 208 further determines a second difference between the transmit clock frequency of the transmit signal and the receive clock frequency of the receive signal. In one embodiment, a second counter 304 is provided which receives the receive clock signal 114B and determines the frequency thereof. In one embodiment, the transmit clock frequency is known, e.g. 100 KHz, and programmed into the processor 208. Alternatively, the transmit clock frequency may be derived from the transmit signal, as discussed above. As was described above, the receive clock frequency is adjusted and a second difference is computed between the transmit clock frequency and the receive clock frequency. The second difference is then compared, such as with comparator 306 or other similar logic, with a second threshold 308. The processor 208 then determines that the electrical connection between the physiological monitoring device and the subject has been interrupted when the second difference deviates from, e.g. exceeds or falls below, a second threshold. In one embodiment, the second threshold is 50 counts of the 60 MHz counter. In this embodiment, a lead off event 110 is then indicated when either the phase difference deviates from the first threshold or the receive clock difference deviates from the second threshold.

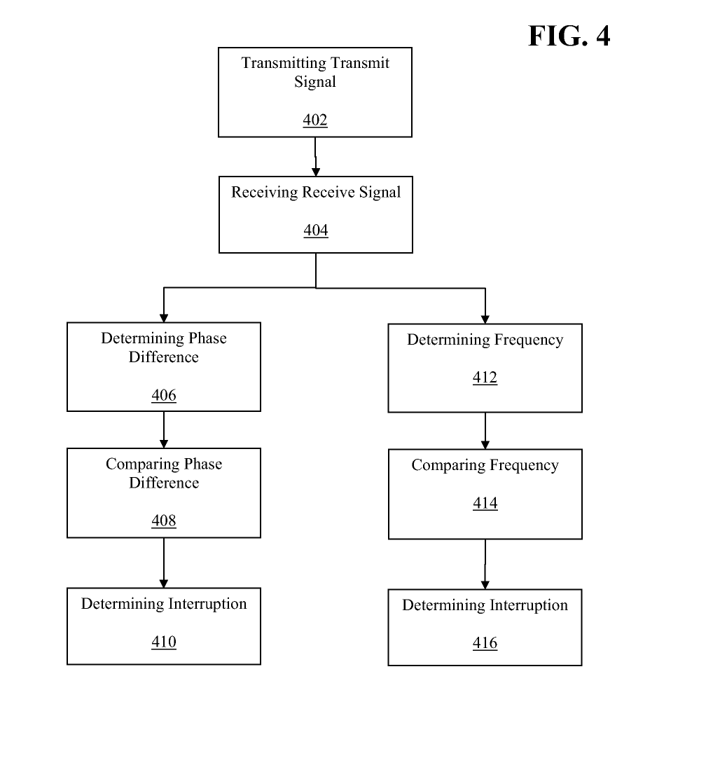

FIG. 4 depicts a flow chart demonstrating operation of the monitoring device 106 of FIG. 1, such as an ECG monitoring device, including a lead off detector 108, according to one embodiment, for detecting an interrupted electrical connection between the physiological monitoring device 106 and a subject 104 being monitored, the physiological monitoring device 106 including a first interface 202A operative to couple the physiological monitoring device 106 with a first electrical lead 112A to be further coupled with the subject 104, the physiological monitoring device 106 further including a second interface 202B operative to couple the physiological monitoring device 106 with a second electrical lead 112B to be further coupled with the subject 104, the first interface 202A, first electrical lead 112A, second interface 202B and second electrical lead 112B forming an electrical connection between the physiological monitoring device 106 and the subject 104. The operation includes: transmitting a transmit signal via the first interface 202A for transmission to the subject 104 via the first electrical lead 112A, the transmit signal being characterized by a transmit phase (Block 402); receiving a receive signal via the second 202B interface, the receive signal being characterized by a receive phase (Block 404); determining a first difference between the transmit phase of the transmit signal and the receive phase of the receive signal (Block 406), such as by counting a number of time intervals elapsed between a leading edge of the transmit signal a leading edge of the receive signal, or alternatively consecutive leading/trailing edges of a combined clock signal, the first difference comprising the counted number of time intervals elapsed; comparing the first difference with a first threshold (Block 408); and determining that the electrical connection between the physiological monitoring device 106 and the subject 104 has been interrupted when the first difference deviates from, such as exceeds or falls below, the first threshold (Block 410).

In alternate embodiment, the operation further includes, where the transmit signal is further characterized by a transmit clock frequency and the receive signal is further characterized by a receive clock frequency: determining a second difference between the transmit clock frequency of the transmit signal and the receive clock frequency of the receive signal (Block 412); comparing the second difference with a second threshold (Block 414); and determining that the electrical connection between the physiological monitoring device 106 and the subject 104 has been interrupted when the second difference deviates from, e.g. exceeds or falls below, a second threshold (Block 416). In embodiments utilizing a combined transmit clock signal and receive clock signal, the frequency may be determined based on consecutive leading edges of the combined clock signal.

It is therefore intended that the foregoing detailed description be regarded as illustrative rather than limiting, and that it be understood that it is the following claims, including all equivalents, that are intended to define the spirit and scope of this invention.

We claim:

1. A method for detecting an interrupted electrical connection between a physiological monitoring device and a subject being monitored, the physiological monitoring device including a first interface operative to couple the physiological monitoring device with a first electrical lead to be further coupled with the subject, the physiological monitoring device further including a second interface operative to couple the physiological monitoring device with a second electrical lead to be further coupled with the subject, the first interface, first electrical lead, second interface and second electrical lead forming an electrical connection between the physiological monitoring device and the subject, the method comprising:
transmitting a transmit signal via the first interface for transmission to the subject via the first electrical lead, the transmit signal being characterized by a transmit phase;
receiving a receive signal via the second interface, the receive signal being characterized by a receive phase;
determining a first difference between the transmit phase of the transmit signal and the receive phase of the receive signal;
comparing the first difference with a first threshold; and
determining that the electrical connection between the physiological monitoring device and the subject has been interrupted when the first difference deviates from the first threshold.

2. The method of claim 1 wherein the physiological monitoring device comprises and electrocardiograph device.

3. The method of claim 1 wherein the determining further determines that the first difference exceeds the first threshold.

4. The method of claim 1 wherein the transmit signal is further characterized by a transmit clock frequency and the receive signal is further characterized by a receive clock frequency, the method further comprising:
determining a second difference between the transmit clock frequency of the transmit signal and the receive clock frequency of the receive signal;
comparing the second difference with a second threshold; and
determining that the electrical connection between the physiological monitoring device and the subject has been interrupted when the second difference deviates from a second threshold.

5. The method of claim 4 wherein the determining of the second difference further comprises deriving the receive clock frequency from the receive signal and deriving the transmit clock frequency from the transmit signal.

6. The method of claim 1 wherein the determining of the first difference further comprises counting a number of time intervals elapsed between a leading edge of the transmit signal a leading edge of the receive signal, the first difference comprising the counted number of time intervals elapsed.

7. The method of claim 1 further comprising:
determining that the electrical connection between the physiological monitoring device and at least one of the first and second electrical leads has been interrupted; and
determining that the electrical connection between at least one of the first and second electrical leads and the subject has been interrupted.

8. A detector for detecting an interrupted electrical connection between a physiological monitoring device and a subject being monitored, the physiological monitoring device including a first interface operative to couple the physiological monitoring device with a first electrical lead to be further coupled with the subject, the physiological monitoring device further including a second interface operative to couple the physiological monitoring device with a second electrical lead to be further coupled with the subject, the first interface, first electrical lead, second interface and second electrical lead forming an electrical connection between the physiological monitoring device and the subject, the detector comprising:
a transmitter operative to transmit a transmit signal via the first interface for transmission to the subject via the first electrical lead, the transmit signal being characterized by a transmit phase;
a receiver operative to receive a receive signal via the second interface, the receive signal being characterized by a receive phase;
a processor coupled with the transmitter and the receiver and operative to determine a first difference between the transmit phase of the transmit signal and the receive phase of the receive signal, the processor being further operative to compare the first difference with a first threshold and determine that the electrical connection between the physiological monitoring device and the subject has been interrupted when the first difference deviates from the first threshold.

9. The detector of claim 8 wherein the physiological monitoring device comprises and electrocardiograph device.

10. The detector of claim 8 wherein the processor is further operative to determine that the first difference exceeds the first threshold.

11. The detector of claim 8 wherein the transmit signal is further characterized by a transmit clock frequency and the receive signal is further characterized by a receive clock frequency, the processor being further operative to determine a second difference between the transmit clock frequency of the transmit signal and the receive clock frequency of the receive signal, compare the second difference with a second threshold, and determine that the electrical connection between the physiological monitoring device and the subject has been interrupted when the second difference deviates from a second threshold.

12. The detector of claim 11 wherein the processor is further operative to derive the receive clock frequency from the receive signal and derive the transmit clock frequency from the transmit signal.

13. The detector of claim 8 wherein the processor is further operative to determine the first difference by counting a number of time intervals elapsed between a leading edge of the transmit signal a leading edge of the receive signal, the first difference comprising the counted number of time intervals elapsed.

14. The detector of claim 8 wherein the processor is further operative to determine that the electrical connection between the physiological monitoring device and at least one of the first and second electrical leads has been interrupted and determine that the electrical connection between at least one of the first and second electrical leads and the subject has been interrupted.

15. A detector for detecting an interrupted electrical connection between a physiological monitoring device and a subject being monitored, the physiological monitoring device including a first interface operative to couple the physiological monitoring device with a first electrical lead to be further coupled with the subject, the physiological monitoring device further including a second interface operative to couple the physiological monitoring device with a second electrical lead to be further coupled with the subject, the first interface, first electrical lead, second interface and second electrical lead forming an electrical connection between the physiological monitoring device and the subject, the detector comprising:
means for transmitting a transmit signal via the first interface for transmission to the subject via the first electrical lead, the transmit signal being characterized by a transmit phase;
means for receiving a receive signal via the second interface, the receive signal being characterized by a receive phase;
means for determining a first difference between the transmit phase of the transmit signal and the receive phase of the receive signal;
means for comparing the first difference with a first threshold; and
means for determining that the electrical connection between the physiological monitoring device and the subject has been interrupted when the first difference deviates from the first threshold.
